(19) United States
(12) Patent Application Publication
(10) Pub. No.: US 2007/0053988 A1
(43) Pub. Date: Mar. 8, 2007

(54) Monodispersed solid lipid particle compositions

(76) Inventors: Audrey Royere, Angers (FR); Jerome Bibette, Paris (FR); Didier Bazile, Angers (FR)

Correspondence Address: YOUNG & THOMPSON, 745 SOUTH 23RD STREET, 2ND FLOOR, ARLINGTON, VA, 22202, US

(21) Appl. No.: 10/575,449

(22) PCT Filed: Sep. 30, 2004
(86) PCT No.: PCT/FR04/02480

§ 371 Date: Jun. 13, 2006

(30) Foreign Application Priority Data
Oct. 13, 2003 (FR) 0311952

(51) Int. Cl. A61K 9/14

(52) U.S. Cl. 424/489

(57) ABSTRACT

A composition includes a monodispersed lipid phase which is dispersed in a continuous aqueous phase, wherein the lipid phase includes at least one crystallizable lipid, at least one active ingredient and at least one compound including two chains of fatty acids and one glycol polyethylene chain. A method for the preparation of the compositions via a simple monodispersed O/W or O/W/O double emulsion is also disclosed.

The present invention relates to monodisperse solid lipid particle compositions comprising active principles.

Solid lipid particle compositions are particularly useful for preparing delivery systems for the administration of one or more active principles to man and animals or for the preparation of vaccines. The administration may take place especially via administration routes such as the oral route, the intravenous route, the subcutaneous route, the intramuscular route, the nasal route, the pulmonary route, the ocular route and the topical route.

Depending on the chosen route of administration, the administration, especially of water-soluble and sparingly water-soluble active principles, poses particular problems.

Thus, in the context of the oral route, it is important to ensure good bioavailability, i.e. a percentage of absorbed active principle, i.e. of active principle present in the blood stream, which is sufficient and whose variability for a given individual, for different dosage intakes and from one individual to another, is satisfactory.

Sparingly water-soluble molecules. In order to be absorbed via the oral route, an active principle must first be dissolved or dispersed in the digestive fluids and then cross the intestinal epithelium.

Means for dissolving or dispersing active principles in aqueous medium are known, such as incorporation into self-emulsifying systems, micelles or liposomes. However, these products are not entirely satisfactory insofar as the objects in suspension obtained are not sufficiently stable on storage and in the digestive fluids.

Suspensions of solid lipid particles make it possible to dissolve and disperse active substances. Specifically, when hot-dispersed in the form of droplets, and then cooled and solidified, these materials can encapsulate active principles that have been dissolved or dispersed beforehand in the molten lipid. The simplicity of the process has made it a serious competitor to systems of polymers coprecipitated as nanoparticles.

Recently, solid lipid nanoparticle suspensions, also known as “SLNs” (solid lipid nanoparticles), have been developed. This type of system has the advantage (i) of being able to be manufactured solvent-free, (ii) of being biodegradable, (iii) free of toxic synthetic residues (SLNs may be prepared from pharmaceutically approved excipients) and (iv) stable with respect to coalescence.

SLNs are stabilized by the presence of surface agents. However, the colloidal stability in suspension during storage and during the preparation process cannot be ensured beyond a certain concentration of dispersed phase, i.e. a few percent by weight (2 to 5%). For higher concentrations, it is difficult to avoid aggregation of the particles.

Thus, document EP 0 605 497 describes an aqueous-phase suspension of lipid particles comprising an active substance. However, the particles obtained according to said document are not monodisperse. Now, the homogeneity of the granulometric distribution of the solid lipid particles in the context of oral administration is an important parameter insofar as the size of the particles conditions (i) the rate of release of the active principle, (ii) the interactions with the gastrointestinal mucosa (given the large developed area of small particles and the bioadhesion properties resulting therefrom), (iii) the degradation by the digestive enzymes, the lipases, which is a surface phenomenon, and (iv) the passage of the particles through the intestinal epithelium. The expected effects of the microencapsulation are (i) an improvement in the dissolution and/or dispersion of the active principle, (ii) protection against degradation by the digestive enzymes and/or the enzymes of intestinal metabolism such as CYP3A4 (in particular for active substances of natural origin), (iii) the possibility of codelivering a P-glycoprotein inhibitor, (iv) where appropriate, protection of the gastrointestinal mucosa when the active principles are irritant, and (v) an increase in lymphatic transportation when the constituents of the particles promote the production of lipoproteins.

Documents U.S. Pat. No. 5,785,976 and U.S. Pat. No. 5,885,486 in the name of Westensen et al. describe suspensions of solid lipid particles.

Document U.S. Pat. No. 6,197,349 in the name of Westensen describes a system for the administration of sparingly soluble active substances by means of particles of supercooled melt (PSM) and suspensions thereof. These particles contain, besides the active substance, only additives to reduce their melting point and also stabilizers, especially amphiphilic stabilizers. They therefore do not contain lipids per se.

Document U.S. 6,207,178 in the name of Westensen describes suspensions of crystalline lipid particles of anisotropic form.

Two processes are mainly used to manufacture these crystallizable emulsions: high-pressure homogenization or intensive mixing, optionally ultrasonication, with heating, followed by cooling. In both cases, the particles obtained have a diameter significantly smaller than one micron.

Water-soluble molecules. The low bioavailability of water-soluble molecules after oral administration is associated with their low diffusion across the biological membranes of the intestinal epithelium. The expected effects of microencapsulation are (i) an increase in the residence time before the absorption window of the gastrointestinal tract (associated with the bioadhesive properties of small particles), (ii) protection against degradation by the digestive enzymes and/or the enzymes of intestinal metabolism such as CYP3A4 (in particular for active substances of natural origin such as peptides, proteins and nucleic acids), (iii) the possibility of codelivering a P-glycoprotein inhibitor, (iv) an increase in the local concentration of the active molecule close to the membrane of the intestinal cells, which promotes diffusion, (v) where appropriate, protection of the gastrointestinal mucosa when the active principles are irritant, and (vi) an increase in lymphatic transportation when the constituents of the particles promote the production of lipoproteins.

A limitation of the process for the preparation of SLNs for hydrophilic molecules lies in their poor encapsulability associated with the low solubility of hydrophilic molecules in oils. To increase the charge content (mass percentage of active principle in the particles), it is possible to encapsulate the active molecule by dissolving it in an aqueous phase and by initially preparing a water-in-oil-in-water double emulsion.

The article by Garcia-Fuentes et al., Colloids and Surfaces B: Biointerfaces, 27 (2002), 159-168, describes the preparation of lipid particles by double emulsion for the oral administration of proteins. However, the protocol uses a solution of tripalmitine (triglyceride) and of lecithin (phospholipids) in methylene chloride. It is therefore not a solvent-free process.

Moreover, emulsification by ultrasonication leads to a calibration of the particles in a size range limited to 0.15-0.5 μm. Finally, the surface agent used in order to give them better stability in digestive fluids is PEG stearate.

However, these particles tend to show substantial and rapid aggregation on storage above a concentration of 5% by weight.

In the context of the nasal route, the expected effects of microencapsulation are (i) an increase in the residence time before the nasal mucosa (associated with the bioadhesive properties of small particles), (ii) protection against degradation by enzymes, (iii) an increase in the local concentration of the active molecule close to the nasal mucosa, which promotes diffusion. The homogeneity of the granulometric distribution of the solid lipid particles in the context of nasal administration is an important parameter insofar as the size of the particles conditions (i) the rate of release of the active principle, (ii) interactions with the nasal mucosa (given the large developed area of small particles and the bioadhesion properties resulting therefrom), (iii) biodegradation, and (iv) the passage of the particles through the nasal mucosa. However, the size range giving the best results in terms of bioavailability and efficacy may be offset relative to the other routes, in particular the oral route.

In the context of the pulmonary route, the granulometric distribution of the administered particles is also important. To reach the pulmonary alveoli, the active molecules must be encapsulated in solid particles that have particular aerodynamic properties. In the current state of knowledge, it is known that a size distribution centered on 3-5 μm allows optimized delivery. Many processes have been proposed to prepare powders whose particles have a narrow size distribution of around 3-5 μm: atomization, precipitation in a nonsolvent, techniques using supercritical carbon dioxide. This technology offers an alternative for producing such particles.

In the context of the subcutaneous administration, lipid microparticles may be prepared for the purpose of proposing an alternative to polymer microspheres. In the article by Reithemeier et al., Journal of Controlled Release 73 (2001) 339-350, a peptide is encapsulated in tripalmitine particles via a double emulsion process. However, in this case also, an organic solvent is used. The homogeneity of the granulometric distribution of the solid lipid particles in the context of subcutaneous administration is an important parameter insofar as the size of the particles conditions (i) the rate of release of the active principle, (ii) the rate of degradation of the particles and their residence time under the skin, and (iii) their interaction with the immune system (macrophages). The constraints are virtually the same for the intramuscular route.

In the context of the intravenous route, the particle size must be less than one micron in order to be compatible with circulation in the blood stream.

Finally, in the context of the preparation of vaccines, the particle size distribution must be adapted to the desired destination of the antigen (antigen-presenting cells) as a function of the route of administration and of the accessibility to immunocompetent cells.

The aim of the invention is thus to propose a process for preparing monodisperse lipid particles comprising at least one active principle, which do not have the drawbacks of the prior art and which are suitable especially for the administration routes indicated above.

A subject of the invention is also a composition that is useful for implementing this process.

Finally, a subject of the invention is the use of these compositions for the preparation of active principle delivery systems.

According to the invention, a composition is thus proposed comprising a monodisperse lipid phase dispersed in a continuous aqueous phase, in which the lipid phase comprises at least one crystallizable lipid, at least one active principle and at least one compound stabilizing the dispersed phase comprising two fatty acid chains and one polyethylene glycol chain.

The term “monodisperse” means a very narrow granulometric distribution of the droplets or globules in the composition. The distribution is considered to be very narrow when the polydispersity is less than or equal to 40% and preferably from about 5% to 30%, for example between 15% and 25%. The polydispersity is then defined as being the ratio of the standard deviation of the curve at the median representing the variation of the volume occupied by the dispersed material as a function of the diameter of the droplets or globules to the mean diameter of the droplets or globules.

The term “solid lipid” or “crystallizable lipid” means a lipid whose melting point is above room temperature, and more precisely lipids with a melting point of from 30 to 95° C. and preferably between 35 and 75° C.

The composition according to the invention is stable for the time required, and especially the time required for the recovery of the dried particles, for example by freeze-drying, therefrom. The term “stable” means that the particles remain individualized and do not aggregate. Advantageously, this stability is conserved even when the concentration of dispersed phase is high, especially when it is greater than 5% by weight.

The composition according to the invention is advantageously compatible with the presence of a high content of dispersed phase. As a result, it allows the preparation of administration systems with a high concentration of active principle. Such administration systems have the advantage of limiting the ingested volume, which promotes patient acceptance.

The content of dispersed phase may thus vary widely according to the intended application. The composition according to the invention may thus especially comprise from 0.01% to 30% by weight of lipid phase.

Moreover, the active principle may be divided between the lipid phase and the aqueous phase during the process. A high content of dispersed phase allows the equilibrium to be displaced towards the lipid phase and to improve the encapsulation yield.

The dispersed lipid phase of the composition may be monophasic or may also comprise a second aqueous phase, referred to as the inner phase, dispersed therein.

In the first case, the emulsion is, at the melting point of the crystallizable lipid, a simple oil/water emulsion. After cooling to the point of solidification of the crystallizable lipid, the dispersed lipid phase transforms into solid lipid particles.

In the second case, the emulsion is, at the melting point of the crystallizable lipid, a water/oil/water double emulsion. Once cooled, solid lipid particles containing aqueous cavities or voids (i.e. containing air or a gas) are obtained as dispersed phase.

In both cases, it is possible to isolate the dispersed phase in order to obtain monodisperse lipid particles containing the active principle(s).

The mean diameter of the dispersed phase in the composition according to the invention is generally between 0.2 and 50 micrometers, preferably between 0.3 and 10 and most particularly between 1 and 6 micrometers.

The composition according to the invention comprises as stabilizer a stabilizing compound bearing two fatty acid chains and one polyethylene glycol chain.

Fatty acid esters of glycerol partially etherified with polyethylene glycol are particularly preferred for use as stabilizer. The fatty acid may especially be a saturated or unsaturated, linear or branched monocarboxylic or dicarboxylic acid containing 8 to 24 carbon atoms. It is preferably a stearate. Advantageously, the stabilizer is a polyethylene glycol ester comprising 25 to 1000, and in particular 32 to 200, polyethylene glycol units.

Preferably, the composition comprises from 0.001% to 30% and preferably from 1% to 10% by weight of stabilizer.

The aqueous phase of the composition according to the invention may comprise, where appropriate, a thickener. The thickening of the continuous phase contributes towards the stabilization of the emulsion. Such thickeners may advantageously be alginic acid salts such as sodium alginate. The thickener may be present in the composition in a proportion of from 0.001% to 10% and preferably from 0.1% to 5% by weight relative to the continuous aqueous phase as a whole.

The aqueous continuous phase may also contain, for example, trehalose, electrolytes, buffers or preserving agents.

The continuous aqueous phase of the composition may also comprise other agents, such as agents for ensuring the isotonicity of the system, cryoprotective agents, buffers or preserving agents.

Among the cryoprotective agents that may especially be mentioned are polyols and electrolytes. In particular, glycerol, mannose, glucose, fructose, xylose, trehalose, mannitol, sorbitol and xylidine or other polyols such as polyethylene glycol are suitable, for example. An electrolyte that may be mentioned is sodium chloride.

The dispersed lipid phase of the composition according to the invention comprises at least one crystallizable lipid.

Among the crystallizable lipids that are especially suitable are natural or synthetic fatty acid mono-, di- or triglycerides, natural or synthetic waxes, wax alcohols and esters thereof, fatty alcohols and esters and ethers thereof, fatty acids and esters thereof, fatty acid glycerides and hydrogenated plant or animal oils, alone or as a mixture.

More particularly, mention may be made of saturated or unsaturated fatty acid mono-, di- or triglycerides containing 8 to 24 carbon atoms, such as glyceride trimyristate, glyceride tripalmitate, glyceride monostearate, cetyl palmitate and hydrogenated olive oil.

Such lipids are commercially available, especially under the following names: Suppocire® DM, Précirol ATO 5, Géléol®, Gélucire® 43/01, Géluciree 62/05, Gélucire® 39/01, Gélucire® 50/02 (Gattefossé), Dynasan 114, Dynasan® 116, Imwitor® 960K, Imwitor® 491, Imwitor® 900P, (Sasol), Oliwax® (QuimDis).

The solid lipid of the dispersed phase has the function of microencapsulating a water-insoluble active principle (this principle may be dissolved or dispersed in the solid lipid) or a water-soluble active principle (this active principle may be dissolved in the inner aqueous phase of the double emulsion or dispersed in the lipid).

Moreover, it may be advantageous for the lipid phase to comprise at least two active principles.

The active principle(s) may be water-soluble or sparingly water-soluble.

Specifically, it is possible, in the case of compositions whose dispersed phase comprises an inner aqueous phase, to convey hydrophilic active principles, alone or combination with the sparingly water-soluble active principles.

According to one specific embodiment of the invention, the lipid phase comprises at least one water-soluble active principle and at least one sparingly water-soluble active principle.

The active principle may especially be a pharmaceutical, veterinary, plant protection, cosmetic or agrifood active principle. Moreover, it may be a detergent, a nutrient, an antigen or a vaccine. It is preferably a pharmaceutical active principle.

Preferably, the pharmaceutical active principle is chosen from the group consisting of antibiotics, hypolipidemiants, antihypertensives, antiviral agents, beta blockers, bronchodilators, cytostatic agents, psychotropic agents, hormones, vasodilators, anti-allergic agents, antalgic agents, antipyretic agents, antispasmodic agents, anti-inflammatory agents, anti-angiogenic agents, antibacterial agents, antiulcer agents, antifungal agents, antiparasitic agents, anti-diabetic agents, antiepileptic agents, antiparkinsonian agents, antimigraine agents, anti-Alzheimer's agents, antiacne agents, antiglaucoma agents, antiasthmatic agents, neuroleptics, antidepressants, anxiolytics, hypnotics, normothymic agents, sedatives, psycho-stimulants, anti-osteoporosis agents, antiarthritic agents, anticoagulants, antipsoriasis agents, hyperglycemiants, orexigenic agents, anorexigenic agents, antiasthenic agents, anticonstipation agents, antidiarrhea agents, antitrauma agents, diuretics, muscle relaxants, enuresis medicaments, erectile dysfunction medicaments, vitamins, peptides, proteins, anticancer agents, nucleic acids, RNA, oligo-nucleotides, ribozymes and DNA.

Moreover, it may prove to be advantageous to combine the active principle(s) with an agent that modifies the oral absorption or an enzyme inhibitor, for example a P-glycoprotein inhibitor or a protease inhibitor.

According to another aspect, the invention relates to a process for preparing a composition comprising a monodisperse lipid phase dispersed in a continuous aqueous phase, in which the lipid phase comprises at least one crystallizable lipid, at least one active principle and a stabilizer, comprising the steps consisting in:
i. introducing the active priniciple(s) into the crystallizable lipid;
ii. dispersing the lipid phase obtained in the aqueous phase in the presence of a stabilizer, to form an emulsion;
iii. subjecting the emulsion obtained to a shear to form a monodisperse emulsion.

According to yet another aspect, the invention relates to a process for preparing a composition comprising a monodisperse lipid phase dispersed in a continuous aqueous phase, in which the lipid phase comprises at least one crystallizable lipid, at least one active principle, a stabilizer and also a dispersed aqueous phase, comprising the steps consisting in:
i. dispersing an aqueous solution comprising the active principle(s) in the lipid melt containing, where appropriate, one or more active principles in the presence of a lipophilic surfactant;
ii. subjecting the emulsion obtained to a shear in order to make it monodisperse;
iii. incorporating the monodisperse emulsion into an aqueous phase in the presence of a stabilizer to form a double emulsion;
iv. subjecting the double emulsion obtained to a shear to form a mondisperse double emulsion.

The controlled shear makes it possible to make the droplets of dispersed phase monodisperse; however, it also makes it possible to control the size of the droplets or globules.

Preferably, the controlled shear is performed by placing the emulsion in contact with a solid surface in motion, the rate gradient characterizing the flow of the emulsion being constant in a direction perpendicular to the solid surface in motion. Such a shear may be produced, for example, in a cell consisting of two concentric cylinders in rotation relative to each other, such as a Couette cell. In this type of cell, the shear is then defined by the number of rotations per minute and the space between the two cylinders.

For details regarding this process, reference is made especially to patent applications WO 97/38787, FR 2 767 064 and WO 01/85319.

The emulsion obtained may then be diluted to the desired concentration.

One or other of these processes also advantageously comprises a cooling step to solidify the dispersed lipid phase.

Thus, according to another aspect, the invention is directed toward monodisperse lipid particles comprising an active principle dissolved or dispersed in a crystallizable lipid, which may be obtained by separation of the continuous aqueous phase of the composition according to the invention.

The aqueous phase may be removed according to one of the means known per se, for instance freeze-drying or atomization.

The composition according to the invention then gives access to monodisperse lipid particles of controllable size.

Thus, the composition according to the invention is particularly useful for preparing systems for delivering water-soluble and/or sparingly water-soluble active principles.

The invention will be understood more clearly with regard to the examples that follow and the figures, which show:

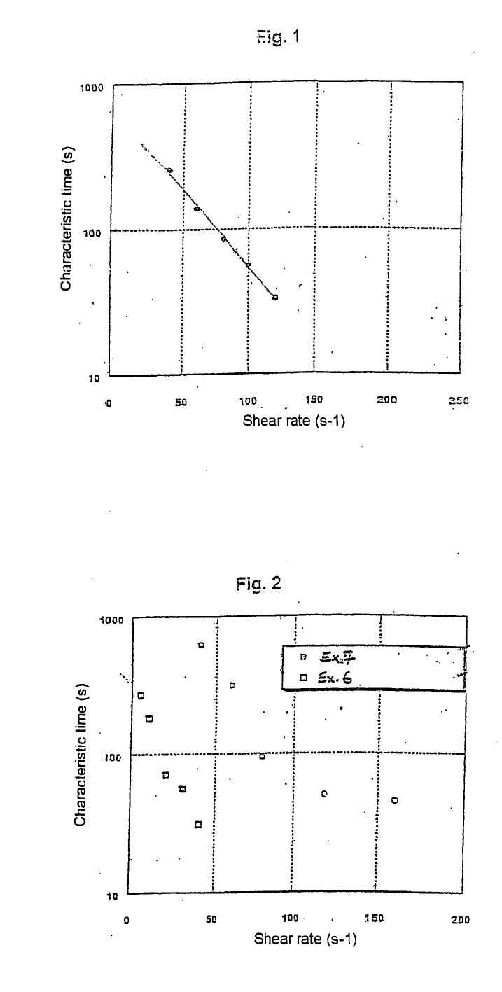

FIG. 1 the characteristic time as a function of the shear rate for the composition of Example 5;

FIG. 2: the characteristic time as a function of the logarithm of the shear rate for the composition of Examples 6 and 7 diluted to 15% by weight of dispersed phase;

FIG. 3: the logarithm of the characteristic time as a function of the shear rate for the composition of Examples 2 and 6, diluted to 15% by weight of dispersed phase;

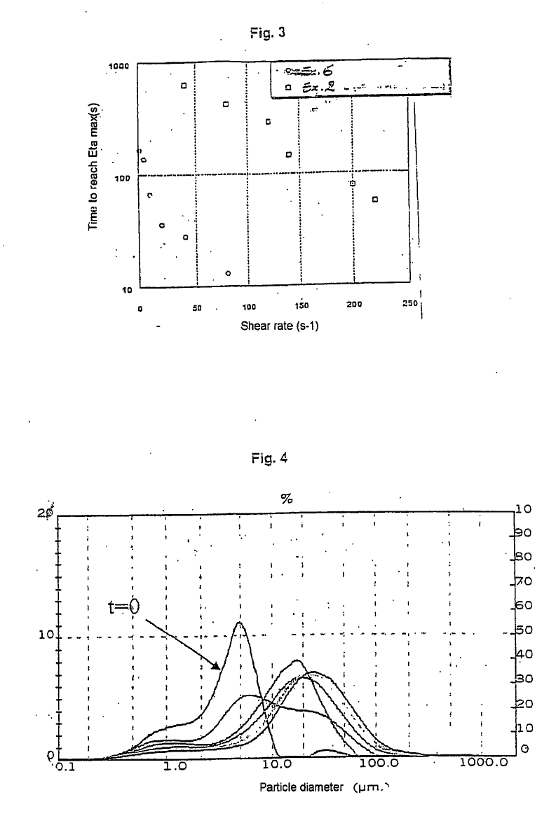

FIG. 4: the change over 30 days of the granulometric distribution of the composition of Example 6;

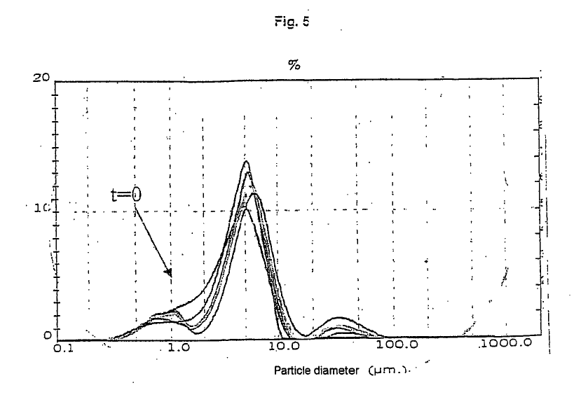

FIG. 5 the change over 30 days of the granulometric distribution of the composition of Example 7.

### EXAMPLES

It is understood that the emulsions to which reference is made hereinbelow are compositions according to the invention, the term being used in order to better highlight the various phases present in the compositions.

The monodisperse emulsions were obtained by firstly preparing an inverse emulsion, which was subjected to a treatment suitable to make it monodisperse. The inverse emulsion was then introduced into an outer aqueous phase to form a double emulsion.

The simple emulsions were obtained by simple emulsification of the fatty phase in the aqueous phase.

### Example 1

Preparation of an Inverse Emulsion

In a container maintained at 65° C. on a water bath, 9.9 grams of PEG-30 dipolyhydroxystearate (30 polyethylene glycol units, Arlacel P135 from Uniqema) and 20.1 g of wax (Suppocire® DM from Gattefossé, a mixture of C₈ to C₁₈ saturated fatty acid glycerides with a melting point of 42 to 46° C.) were mixed together. 70 g of an aqueous NaCl solution (0.6 g/l, 0.4M) preheated to 65° C. were dispersed in this fatty phase. The emulsion obtained, of water-in-oil type, contained 70% by weight of dispersed phase.

The emulsion obtained was then introduced into a Couette device heated to 65° C. and subjected to a shear defined by a spin speed of 400 rpm for an injection rate of 7 ml/min corresponding to an injection speed of 0.7.

The emulsion obtained was calibrated with a mean size of the dispersed phase of 400 nanometers, and was stored in an oven at 70° C.

### Example 2

Double Emulsion

40 g of the calibrated inverse emulsion obtained in Example 1 were diluted in 60 g of wax (Suppocire® DM, mixture of C₈ to C₁₈ saturated fatty acid glyceride) preheated to 60° C.

6 g of the dilute calibrated inverse emulsion thus obtained were then incorporated, still at 65° C., into 4 g of an aqueous phase composed of water and 8% of a stabilizer (Gélucire® 4414 from Gattefossé, defined mixture of mono-, di- and triglycerides and of mono-, di- and triesters of polyethylene glycol and of fatty acids), 11.5% of glucose and 0.5% of sodium alginate HM120L, from Aldrich) to form a double emulsion. This premix contained 60% by weight of dispersed phase.

The premix was subjected to a shear in a Couette device at 150 rpm at an injection speed of 0.7 at a temperature of 65° C. The emulsion obtained was calibrated with a mean diameter of the dispersed phase centered about 4 μm.

After emulsification, the emulsion may be hot-diluted in an aqueous solution containing 11.5% glucose, to the desired lipid phase content. After dilution, the emulsion was stored at 5° C.

### Example 3

Double Emulsion

The inverse emulsion obtained in Example 1 was incorporated after dilution as in Example 2 into an aqueous phase containing only 5% stabilizer (Gélucire® 4414) and 0.2% sodium alginate.

The premix obtained as in Example 2 was then sheared in a Couette device at 75 rpm at an injection speed of 0.7. The double emulsion obtained was calibrated, the mean size of the dispersed phase being 6.86 μm.

### Example 4

Double Emulsion

A double emulsion was prepared as in Example 2, except that the aqueous phase contained as stabilizer 4% of PEG-150 distearate (Stepan® PEG6000 DS from Stepan) and 11.5% glucose.

The premix was sheared at 200 rpm at an injection speed of 0.7 to give a double emulsion whose dispersed phase has a mean diameter centered about 4 μm.

### Example 5

Simple Emulsion

5-1 6 g of wax heated on a water bath at 60° C. (Suppocire® DM, mixture of C₈ to C₁₈ saturated fatty acid glycerides) were incorporated into 4 g of aqueous solution containing 8% by weight of stabilizer (Gélucire® 4414).

The premix was then sheared in a Couette device at 600 rpm at an injection speed of 0.7 to give a simple emulsion with a mean diameter centered on 1 μm.

5-2 6 g of wax (Suppocire® DM, mixture of C₈ to C₁₈ saturated fatty acid glycerides) were incorporated into 4 g of aqueous solution containing 8% by weight of stabilizer (Gélucire® 4414) and 0.5% sodium alginate.

The premix was then sheared in a Couette device at 150 rpm at an injection speed of 0.7 to give a simple emulsion whose dispersed phase has a mean diameter centered on 6 μm.

### Example 6

Simple Emulsion

36.5 g of wax (Suppocire® DM, mixture of C₈ to C₁₈ saturated fatty acid glycerides) were incorporated into 13.5 g of aqueous solution containing 14.5% by weight of stabilizer (Gélucire® 4414), 4.3% by weight of trehalose and 0.85% by weight of sodium alginate as in the above example.

The premix was then sheared in a Couette device at 200 rpm at an injection speed of 0.7 at 58° C. to give a simple emulsion whose dispersed phase has a mean diameter centered on 4.8 μm.

### Example 7

Simple Emulsion

36.5 g of wax (Suppocire® DM, mixture of C₈ to C₁₈ saturated fatty acid glycerides) were incorporated into 13.5 g of aqueous solution containing 6.6% by weight of stabilizer (PEG-150 distearate; Stepan® PEG6000 DS from Stepan) and 4.3% of trehalose, as in Example 5.

The premix was then sheared in a Couette device at 200 rpm at an injection speed of 0.7 at a temperature of 57° C. to give a simple emulsion whose dispersed phase has a mean diameter centered on 4.8 μm.

Stability of the Emulsions

The emulsions prepared were characterized in terms of stability.

Stability of the various formulations was evaluated especially by means of rheological studies. The controlled flow of the emulsions was studied in a rheometer with cone/plate geometry (RS2, Ademtec) having the following characteristics:
diameter: 50 mm,
cone angle: 0.04 rad,
gap: 0.0453 mm.

The temperature of the rheometer is kept constant at 25° C.

The emulsions were prepared one day in hand according to the above examples, diluted to the desired lipid-phase fraction, and then divided into aliquots in 5 ml pill bottles in order for each sample to undergo the same process before the rheological study. These samples were stored at 5° C.

Before each measurement, the pill bottle was shaken gently (upturned two or three times) and the emulsion was then poured cautiously onto the plate.

An increase in viscosity after a characteristic time is found for each of the emulsions studied. This viscosity increase is accompanied by the appearance of the creamy texture, which is noticed after manual shaking. The characteristic time taken is that corresponding to the maximum viscosity.

A change in texture is also observed by microscope. The texture of the emulsions is characterized by the presence of globules of substantially equal size. During the viscosity increase, the globules aggregate to form irregular and anisotropic clusters of dispersed phase.

This phenomenon is irreversible. It is assumed that these clusters condition the “jamming” phenomenon during flow.

The characteristic time depends on the shear rate (FIG. 1). Specifically, it is observed that the characteristic time decreases as the shear rate increases.

The characteristic time follows an exponential dependence of the type whose point T is equal to τ₀ X (E^{-γ/γc}) where 1/γ_{c} is the characteristic time of the phenomenon. Thus, when the logarithm of the characteristic time is placed as a function of the shear rate, a curve is obtained whose intercept at zero shear indicates the lifetime of the material at rest, i.e. under storage conditions without shear.

This curve is shown in FIG. 2 for the emulsion of Examples 5 and 6, diluted to 15% by weight of dispersed phase, respectively. These emulsions differ mainly in the nature of the stabilizer used.

It is found that the characteristic time is longer for the emulsion of Example 6. This observation makes it possible to conclude that stabilization of the dispersed phase with a compound containing a long PEG chain (150 PEG units) affords better stability of the emulsion. On the other hand, the emulsion stabilized with a compound containing a shorter PEG chain (32 PEG units) has a shorter characteristic time and thus lower stability.

Secondly, it is found that the characteristic time of a simple emulsion is shorter than that of a comparable double emulsion. FIG. 3 shows the characteristic time as a function of the shear rate for the emulsions of Examples 2 and 5, respectively, diluted to 15% of dispersed phase. These emulsions are stabilized with the same compound. The characteristic time values indicate that a double emulsion is more stable than a comparable simple emulsion. Thus, it appears that the presence of a dispersed aqueous phase in the dispersed lipid phase of the emulsion stabilizes the emulsion and, as a result, prolongs the lifetime of the system.

In a complementary test, the stability of the granulometric distribution of the lipid particles in the suspension was observed.

The granulometric analysis was performed using a MasterSizer S laser granulometer from Malvern with a 150 ml cell, assuming the refractive index of the dispersed phase corresponding to that used in the 3OJD presentation.

FIGS. 4 and 5 thus show the granulometric distributions of the emulsions of Examples 5 and 6, respectively, the mean globule diameter of which was centered about 4 μm, measured at different time intervals. Between the measurements, the emulsions, diluted to 5% of dispersed phase, were stored at 5° C.

It is found that the emulsion prepared with a stabilizer containing 150 PEG units has greater stability than the emulsion obtained with a stabilizer containing 32 PEG units.

### Example 8

Removal of the Aqueous Phase of the Emulsion by Freeze-Drying:

After emulsification, the calibrated emulsion obtained in Examples 2 to 7 hot-diluted (typically at 65° C.) in an aqueous solution containing 11.5% by weight of trehalose and 0.25% by weight of sodium hyaluronate, to a proportion of 5% by weight of lipid phase.

The emulsion is then frozen and placed in a freeze-dryer (Lyovac GT2 Steris freeze-drying machine and Phoenix C75P Thermo Haake cryostat).

Calibrated lipid particles are obtained.

The particles obtained do not show any aggregation when observed by optical microscopy (redispersed in an aqueous solution containing a surfactant).

1-27. (canceled)

28. A composition comprising a monodisperse lipid phase dispersed in a continuous aqueous phase, in which the lipid phase comprises at least one crystallizable lipid, at least one active principle and at least one compound stabilizing the dispersed phase comprising two fatty acid chains and one polyethylene glycol chain.

29. The composition as claimed in claim 28, in which an inner aqueous phase is dispersed in the dispersed lipid phase.

30. The composition as claimed in claim 28, in which the dispersed lipid phase has a mean diameter of between 0.3 and 10 micrometers.

31. The composition as claimed in claim 28, comprising 0.01% to 30% by weight of lipid phase.

32. The composition as claimed in claim 28, comprising 0.001% to 30% by weight of compound for stabilizing the dispersed phase.

33. The composition as claimed in claim 28, in which the polyethylene glycol chain comprises 25 to 1000 ethylene glycol units.

34. The composition as claimed in claim 28, in which the continuous aqueous phase further comprises 0.001% to 10% by weight of a thickener.

35. The composition as claimed in claim 34, in which the thickener is an alginic acid salt.

36. The composition as claimed in claim 28, in which the crystallizable lipid is chosen from natural or synthetic fatty acid mono-, di- or triglycerides, natural or synthetic waxes, wax alcohols and esters thereof, fatty alcohols and esters and ethers thereof, fatty acids and esters thereof, fatty acid glycerides and hydrogenated plant or animal oils, alone or as a mixture.

37. The composition as claimed in claim 36, in which the crystallizable lipid is a C₁₂-C₁₈ mono-, di- or triglyceride.

38. The composition as claimed in claim 28, in which the continuous aqueous phase comprises a cryoprotective agent.

39. The composition as claimed in claim 38, in which the cryoprotective agent is a polyol or a salt.

40. The composition as claimed in claim 28, in which the lipid phase comprises at least two active principles.

41. The composition as claimed in claim 28, in which the lipid phase comprises at least one water-soluble active principle.

42. The composition as claimed in claim 28, in which the lipid phase comprises at least one sparingly water-soluble active principle.

43. The composition as claimed in claim 28, in which the lipid phase comprises at least one water-soluble active principle and at least one sparingly water-soluble active principle.

44. The composition as claimed in claim 28, in which the active principle is chosen from the group of pharmaceutical, veterinary, plant-protection, cosmetic and agrifood active principles.

45. The composition as claimed in claim 28, in which the active principle is a detergent, a nutrient, an antigen or a vaccine.

46. The composition as claimed in claim 28, in which the water-soluble pharmaceutical active principle is chosen from the group consisting of antibiotics, hypolipidemiants, antihypertensives, antiviral agents, beta blockers, bronchodilators, cytostatic agents, psychotropic agents, hormones, vasodilators, antiallergic agents, antalgic agents, antipyretic agents, antispasmodic agents, anti-inflammatory agents, anti-angiogenic agents, antibacterial agents, antiulcer agents, antifungal agents, antiparasitic agents, antidiabetic agents, antiepileptic agents, antiparkinsonian agents, antimigraine agents, anti-Alzheimer's agents, antiacne agents, antiglaucoma agents, antiasthmatic agents, neuroleptics, antidepressants, anxiolytics, hypnotics, normothymic agents, sedatives, psychostimulants, anti-osteoporosis agents, antiarthritic agents, anticoagulants, antipsoriasis agents, hyperglycemiants, orexigenic agents, anorexigenic agents, antiasthenic agents, anticonstipation agents, antidiarrhea agents, antitrauma agents, diuretics, muscle relaxants, enuresis medicaments, erectile dysfunction medicaments, vitamins, peptides, proteins, anticancer agents, nucleic acids, RNA, oligonucleotides, ribozymes and DNA.

47. The composition as claimed in claim 28, in which the active principle(s) is(are) combined with an agent that modifies the oral absorption or an enzyme inhibitor.

48. The composition as claimed in claim 47, in which the enzyme inhibitor is a P-glycoprotein inhibitor or a protease inhibitor.

49. A process for preparing a composition comprising a monodisperse lipid phase dispersed in a continuous aqueous phase, in which the lipid phase comprises at least one crystallizable lipid, at least one active principle and a stabilizer, comprising the steps consisting in:
i. introducing the active priniciple(s) into the crystallizable lipid;
ii. dispersing the lipid phase obtained in the aqueous phase in the presence of a stabilizer, to form an emulsion;
iii. subjecting the emulsion obtained to a shear to form a monodisperse emulsion.

50. A process for preparing a composition comprising a monodisperse lipid phase dispersed in a continuous aqueous phase, in which the lipid phase comprises at least one crystallizable lipid, at least one active principle, a stabilizer and also a dispersed aqueous phase, comprising the steps consisting in:
dispersing an aqueous solution comprising the active principle(s) in the lipid melt containing, where appropriate, one or more active principles in the presence of a lipophilic surfactant;
i. subjecting the emulsion obtained to a shear in order to make it monodisperse;
ii. incorporating the monodisperse emulsion into an aqueous phase in the presence of a stabilizer to form a double emulsion;
iii. subjecting the double emulsion obtained to a shear to form a mondisperse double emulsion.

51. The process as claimed in claim 49, further comprising a cooling step to solidify the dispersed lipid phase.

52. A process for preparing monodisperse lipid particles comprising at least one active principle, comprising the removal of the aqueous phase of a composition prepared according to the process of claim 49.

53. A process for preparing monodisperse lipid particles comprising at least one active principle, comprising the removal of the aqueous phase of a composition prepared according to the process of claim 50.

54. The process as claimed in claim 53, in which the aqueous phase is removed by freeze-drying, if necessary after diluting the composition in a solution containing a cryoprotective agent.
